(19) United States
(12) Patent Application Publication
(10) Pub. No.: US 2025/0205172 A1
(43) Pub. Date: Jun. 26, 2025

(54) HEAT PATCH CAPABLE OF TARGETED DRUG DELIVERY BASED ON MONROE EFFECT

(71) Applicant: Longxi Cheezheng Medicinal Materials Co., Ltd., Dingxi City (CN)

(72) Inventors: Ruoqiong CHENG, Dingxi City (CN); Yue MA, Dingxi City (CN); Bo YANG, Dingxi City (CN); Jing HAN, Dingxi City (CN); Rongrong HE, Dingxi City (CN)

(21) Appl. No.: 18/425,309

(22) Filed: Jan. 29, 2024

(30) Foreign Application Priority Data
Dec. 20, 2023 (CN) 202311763659.8

(51) Int. Cl. A61K 9/70

(52) U.S. Cl. CPC A61K 9/7084

(57) ABSTRACT

A heat patch capable of targeted drug delivery based on a Monroe effect include: a fixation layer, a heating layer and a drug layer that are arranged in sequence, where a heating assembly is arranged in the heating layer, a heat collection cavity that is provided with an opening facing the drug layer is provided in a side, close to the drug layer, in the heating layer, a drug is contained at a portion, corresponding to the opening of the heat collection cavity, in the drug layer, and the heat collection cavity has a shape of any or a combination of a semicircle, a cone and a semi-elliptic shape. The heat collection cavity is provided, and the heat collection cavity fully exerts the Monroe effect, effectively collects heat released in a chemical reaction in the heating layer, and transfers heat to the drug in a concentrated manner.

### TECHNICAL FIELD

The present disclosure relates to the technical field of medical supplies, in particular to a heat patch capable of targeted drug delivery based on a Monroe effect.

### BACKGROUND ART

An existing heat patch is mainly composed of a release liner 1, an adhesive layer 2, a drug layer 3 and a heating layer 4 as shown in FIG. 1. The drug layer and heating layer are arranged in two connected non-woven bags 5 respectively. A gelatin layer is used to make the drug layer thermally adhere to the skin of a patient. A raw material layer is an iron powder vermiculite heating bag composed of iron, vermiculite, activated carbon, inorganic salts, etc., and can have an exothermic reaction under the action of oxygen in the air. Since the non-woven bag is uniform, leakproof and desirable in air permeability, air can flow into the heating layer smoothly, and cause an exothermic reaction between polymers. The heat released by the heating layer heats the drug layer after being transferred to the same, such that drug penetrates into subcutaneous tissue through pores of the skin of the patient, and then reaches a focus through a blood circulation system, thus achieving the therapeutic effect. The heat patch combines the physiotherapeutic effect of the drug with the hyperthermia effect to relieve pain for the patient.

However, the existing heat patch still has the following technical problems. At first, heat energy released by the heating layer in the existing heat patch can heat up ointment and speed up volatilization and permeation of Chinese medicinal components in the ointment. However, the heat energy suffers serious loss when dispersively transferred to the ointment, and has low utilization efficiency, which slow down the permeation and absorption of the drug, and reduce the therapeutic effect. In addition, due to the low utilization efficiency of the heat energy in the heat patch, Chinese medicinal ointment in the existing heat patch is added excessively for improving the therapeutic effect, but cannot be absorbed fully after the heat energy in the heating layer is completely released every time. As a result, low utilization efficiency of the ointment and waste of abundant medicinal materials are caused. Thirdly, the drug of the existing heat patch is delivered in a decentralized manner, and needs to be completely absorbed through skin pores before being circulated to the focus with the blood, resulting in a low therapeutic effect.

To this end, it is urgent to develop a heat patch that has a desirable therapeutic effect, a low use amount of ointment and a low cost and is capable of targeted drug delivery, which can effectively make up for the shortcomings of the prior art, further effectively improve the therapeutic effect and reduce the therapeutic cost. In addition, the heat patch is of great significance for treatment of chronic diseases, such as rheumatic arthralgia, lumbago and leg pain, lumbar muscle strain, soft tissue injury, stomach cold, abdominal pain and dysmenorrhea.

### SUMMARY

An objective of the present disclosure is to provide a heat patch capable of targeted drug delivery based on a Monroe effect, so as to solve a technical problem of an existing heat patch that heat energy suffers a great loss when dispersively transferred to Chinese medicinal ointment.

In order to achieve the above objective, the present disclosure adopts the following technical solution: a heat patch capable of targeted drug delivery based on a Monroe effect includes: a fixation layer, a heating layer and a drug layer that are arranged in sequence, where a heating assembly is placed in the heating layer, a heat collection cavity that is provided with an opening facing the drug layer is provided in a side, close to the drug layer, in the heating layer, and a drug is contained at a portion, corresponding to the opening of the heat collection cavity, in the drug layer.

This solution is based on a principle as follows:

In this solution, the fixation layer is configured to fix the heat patch on an affected part of a patient, such that the drug layer can come into contact the affected part of the patient conveniently and exert a therapeutic effect. The heating layer is configured to release heat energy after the heat patch is fixed on the affected part of the patient, such that drug absorption of the affected part of the patient can be promoted. In this solution, the heat collection cavity is provided in the side, close to the drug layer, of the heating layer, and the heat collection cavity fully exerts the Monroe effect. That is, a recess is provided in a vertically dispersive heat transfer path, and after passing through the recess, the heat can converge into a bunchy heat flow that has a high speed and a high pressure at a center line of the recess, such that a heat utilization effect is improved in an order of magnitude. Especially, under the condition of less surrounding heat, the heat collection cavity can collect the heat around the heat collection cavity into bunchy heat and transfer same to the drug layer, such that the drug can come into contact skin of a patient in a bunchy or spotted shape, heat utilization efficiency can be effectively improved, and a high temperature perception threshold and cryogenic burn risk of the patient can be further reduced. In addition, the heat collection cavity collects the heat in the heating layer and transfers same to the drug layer in a concentrated manner, such that a heat energy transfer loss can be effectively reduced and the utilization efficiency of heat energy in the heating layer can be improved. In this solution, the drug is contained at the portion, corresponding to the opening of the heat collection cavity, in the drug layer, and the heat energy collected in the heat collection cavity is transferred to the drug in a concentrated manner accordingly, such that the absorption efficiency of the drug is effectively improved and the therapeutic effect is further improved.

This solution has advantages as follows:
1. In the prior art, heat in a heating layer is dispersively transferred to a drug layer, and suffers a great heat loss. In this solution, the heat collection cavity is provided in the side, close to the drug layer, of the heating layer, such that the heat released from the heating layer is effectively collected, the utilization efficiency of heat energy is improved and a cost of heating materials in the heating layer is reduced.
2. In the prior art, drug absorption efficiency is reduced due to the heat energy loss. According to the solution, the heat collection cavity is provided, and the heat collection cavity fully exerts the Monroe effect, effectively collects heat released in a chemical reaction in the heating layer, and transfers heat to the drug in a concentrated manner. In this way, heating and volatilization release efficiency of the drug can be promoted, and absorption efficiency of the drug can be improved accordingly. In addition, concentrated heat transfer can also make drug volatilization and absorption more concentrated, the targeted drug delivery is effectively implemented, and the therapeutic effect of the drug is improved accordingly.
3. In this solution, the drug is contained at the portion, corresponding to the heat collection cavity, in the drug layer, such that the targeted drug delivery can be implemented, a use amount of the drug for heat energy treatment in the heating layer can be further effectively reduced one time, and a cost of the drug can be effectively reduced. An existing heat patch suffers waste of medicinal materials due to dispersive drug absorption caused by dispersive heat transfer. According to the solution, the heat energy is transferred by the heat collection cavity to continuously act on the drug, such that the same amount of heat has a better heating effect, the utilization efficiency of the drug is improved, waste of the drug is effectively reduced, the cost of the drug is further reduced, and efficacy of the drug can be further improved.
4. According to this solution, the drug layer is arranged, and the drug is contained in the drug layer. The heat in the heating layer mainly comes into contact with the skin of the patient through the drug after the heat is collected in the heat collection cavity, such that the targeted drug delivery can be implemented, a contact area between the skin of the patient and the drug can be reduced, and the drug can be cleared conveniently after being used. In addition, according to this solution, a barrier is formed between the heating layer and the skin of the patient at a position other than the position where the drug is contained in the drug layer, such that a direct contact between the skin of the patient and the heating layer can be effectively avoided, possible cryogenic burns caused by direct transfer of the heat of the heating layer to the skin of the patient can be effectively avoided, and use safety of the heat patch can be improved.

Preferably, the heat collection cavity has a shape of any or a combination of a semicircle, a cone and a semi-elliptic shape.

This solution has beneficial effects as follows: due to adoption of the above arrangement, the semicircle or the cone is more conducive to bunchy heat collection, such that the heat utilization efficiency can be effectively improved. The bunchy heat is transferred to the drug, such that Chinese medicinal substances in the drug also permeate into the skin of the patient in bunches. In this way, the targeted drug delivery effect of the drug is improved, the contact area between a heated drug and the skin of the patient can be further reduced, the cryogenic burn risk is further reduced and the use safety of the heat patch is further improved.

Preferably, the heat collection cavity is made of any of stainless steel, high-carbon steel, engineering plastics, memory alloys and non-woven fiber elastic cotton.

This solution has beneficial effects as follows: due to adoption of the above arrangement, the heat in the heating layer can be collected by the heat collection cavity and then transferred to the drug layer conveniently, such that the utilization efficiency of heat energy and the therapeutic effect of the drug are improved.

Preferably, the ratio between a height h and a diameter d of the heat collection cavity is 0.3-1, and the diameter d≤10 cm.

This solution has beneficial effects as follows: due to adoption of the above arrangement, the heat collection effect of the heat collection cavity can be effectively improved conveniently. Through long-term experiments, the applicant finds that the shape and the size of the heat collection cavity can affect the heat collection effect. If the ratio of the height to the diameter is too small, the heat collection effect can be reduced since a cross section of the heat collection cavity is closer to a plane. If the ratio of the height to the diameter is too large, inconvenient use is caused since a thickness of the heat patch can be increased, and in the case of the excessively height, a poor heat transfer effect can be caused, and it takes too long time for heating the drug layer with the heat. In addition, the applicant also finds that if the diameter is too large, the cavity is too large, the heat collection effect of the heat generated by the heating materials can be worsened, specifically a surface temperature of the drug is reduced, and the therapeutic effect of the drug is reduced accordingly.

Preferably, the drug layer is provided with a drug cavity that is configured to contain the drug corresponding to the heat collection cavity, the drug cavity is a blind hole, and the drug cavity is located in a side, far away from the heating layer, of the drug layer.

This solution has beneficial effects as follows: due to adoption of the above arrangement, the drug can be collected in the drug cavity conveniently, such that the condition that the drug enters the insulation layer and influences the heat collection and heat transfer effects and reduces the heat utilization efficiency and the therapeutic effect can be effectively avoided.

Preferably, the drug contained in the drug cavity is any or a combination of drug powder, a pill, ointment and a drug liquid.

This solution has beneficial effects as follows: due to adoption of the above arrangement, the patient can conveniently select different forms of drugs with the heat patch of this solution according to illness for targeted treatment. In specific use, the patient first fills the drug cavity with the drug, and then fixes the heat patch by sticking one side of the drug layer of the heat patch to the skin of the patient, such that fixed-point targeted treatment of the affected part can be implemented.

Preferably, a heat dissipation hole is provided between the drug cavity and the heat collection cavity.

This solution has beneficial effects as follows: due to adoption of the above arrangement, the heat in the heat collection cavity can be quickly transferred to the drug in the drug cavity conveniently, such that the drug can be quickly heated, the absorption efficiency of the drug can be improved, and the targeted drug delivery can be implemented.

Preferably, the heating layer includes a heating sleeve located peripherally and the heating assembly located inside, the heating assembly is any of an electric heating sheet, a magnetic heating sheet or an iron powder vermiculite heating bag, and the heating sleeve and the fixation layer are provided with replacement openings.

This solution has beneficial effects as follows: due to adoption of the above arrangement, the heating assembly can be replaced conveniently, such that the heat patch can be used repeatedly, and the therapeutic cost for the patient can be effectively reduced.

Preferably, the heating assembly is the iron powder vermiculite heating bag, an insulation layer is arranged on a side, far away from the drug layer, of the fixation layer, the insulation layer includes a fixed end connected to the fixation layer and a windable free end, and the fixation layer is provided with a fixation member that detachably fixes the free end of the insulation layer near the fixed end.

This solution has beneficial effects as follows: due to adoption of the above arrangement, the heating assembly is the iron powder vermiculite heating bag, and limited time targeted treatment with the drug can be implemented by controlling a use amount of the iron powder vermiculite heating bag. The insulation layer and the fixation member are arranged, and the insulation layer includes two states of wound and unwound isolation since the fixation member fixes the insulation layer in different fixation states. Specifically, in an early use stage of the heat patch, the insulation layer is wound to facilitate air in contact the iron powder vermiculite heating bag through the fixation layer and the heating sleeve, such that the iron powder vermiculite heating bag reacts with oxygen in the air to release heat, and the heat is transferred to the drug in a bunchy shape for targeted treatment. However, when the heating assembly is too hot and makes the patient uncomfortable (for example, some temperature-sensitive patients have poor temperature tolerance), winding fixation of the insulation layer by the fixation member is released, the insulation layer is unwound and closely attached to an outer side surface of the heat patch, and in this case, the insulation layer is in an unwound isolation state. In this way, the oxygen in the air can be isolated effectively from entering the heating layer, the chemical exothermic reaction of the iron powder vermiculite heating bag can be blocked, and heat release is reduced and physical perception of the patient is relieved. According to this solution, the application scope of the heat patch is effectively expanded by arranging the insulation layer.

Preferably, an adhesive coating and a release liner are sequentially arranged on a side, facing the drug layer, of the fixation layer.

This solution has beneficial effects as follows: due to adoption of the above arrangement, the heat patch can be stuck and fixed to the affected part of the patient conveniently. In specific use, the patient first tears off the release liner, then fills the drug cavity with the drug, then sticks one side of the drug layer of the heat patch to the skin of the patient, and presses the fixation layer to make the adhesive coating to be stuck to the skin of the patient. After fixing the heat patch, the heating assembly releases heat, the heat is collected in a bunchy shape by the heat collection cavity and transferred to the drug in the drug layer, then the drug permeates into the affected part of the patient, and targeted treatment with the drug is implemented.

Preferably, the drug layer is made of heat insulation materials.

This solution has beneficial effects as follows: due to adoption of the above arrangement, heat that is directly transferred to the skin of the patient through the materials of the drug layer from the heating layer can be further reduced conveniently, and the cryogenic burn risk can be further reduced. In addition, heat insulation cotton has a desirable heat effect, such that the heat loss can be effectively avoided, the heat utilization efficiency can be provided and a permeation effect of the drug can be improved.

Preferably, the heating assembly is a heat insulation bag and the fixation layer is made of a non-woven material.

This solution has beneficial effects as follows: due to adoption of the above arrangement, ventilation is facilitated, such that the air can quickly enter the heating layer to be quickly heated, work duration of the drug is effectively shortened, and the therapeutic effect is improved. In addition, the non-woven fabric material is also convenient to clean, such that cleaning and reuse of the heat patch can be implemented.

Preferably, one or several heat collection cavities are provided, one heat collection cavity is located in a middle of the heating layer of the heat patch, and the several heat collection cavities are distributed in a matrix.

This solution has beneficial effects as follows: due to adoption of the above arrangement, different numbers of heat collection cavities can be selected according to concentration or dispersion of acupuncture points of the affected part of the patient. For example, when the acupuncture points are concentrated, a heat patch that has one heat collection cavity can be selected, and in this case, the heat collection cavity is located at a center of a heating layer (that is, a center of the heat patch). Heat of the heating layer can be collected through the heat collection cavity, surrounding low heat can be collected to heat the drug in a concentrated manner, and the heated drug can be delivered to the affected part in a concentrated manner. A surface temperature of the drug can be effectively increased even in a low-heat environment, a therapeutic effect of targeted drug delivery can be achieved, the therapeutic effect can be effectively improved by using low-temperature heat, and the heat utilization efficiency can also be improved. When the acupuncture points at the affected part of the patient are dispersive, a heat patch that has several heat collection cavities of matrix distribution can be selected, such that the several heat collection cavities can facilitate dispersive drug delivery of matrix distribution after collecting heat separately, targeted drug delivery to a plurality of acupuncture points can be implemented, and the therapeutic effect can be improved.

### BRIEF DESCRIPTION OF THE DRAWINGS

FIG. 1 is a schematic structural diagram of an existing heat patch.

FIG. 2 is a schematic structural diagram of the heat patch capable of targeted drug delivery based on a Monroe effect in Example 1 of the present disclosure.

FIG. 3 is a structural comparison diagram of sections of different heat collection cavities according to an example of the present disclosure (A denotes a conical heat collection cavity, B denotes a hemispherical heat collection cavity, C denotes a semi-elliptic heat collection cavity, D denotes a combined conical heat collection cavity, E denotes a cylindrical heat collection cavity, and arrows indicate a direction of an opening of the heat collection cavity).

FIG. 4 is a front view (in a state with a release liner torn off) of a heat patch capable of targeted drug delivery based on a Monroe effect according to Example 9 of the present disclosure.

FIG. 5 is a sectional view (that is, a sectional view of a heat patch capable of targeted drug delivery based on a Monroe effect in Example 9) along line A-A in FIG. 4.

FIG. 6 is an enlarged view of part A in FIG. 5.

FIG. 7 is a sectional view of a heat patch capable of targeted drug delivery based on a Monroe effect in Example 10 of the present disclosure.

FIG. 8 is an enlarged view of portion B in FIG. 7.

FIG. 9 is a sectional view of a heat patch capable of targeted drug delivery based on a Monroe effect in Example 11 of the present disclosure.

FIG. 10 is an enlarged view of part C in FIG. 9.

FIG. 11 is a rear view of a heat patch capable of targeted drug delivery based on a Monroe effect in Example 13 of the present disclosure.

### DETAILED DESCRIPTION OF THE EMBODIMENTS

Detailed description will be made below with reference to specific embodiments.

The reference numerals in accompanying drawings of the description are as follows: release liner 1, adhesive layer 2, drug layer 3, drug cavity 31, heat dissipation hole 32, heating layer 4, heating sleeve 41, heating assembly 42, heat collection cavity 43, non-woven bag 5, fixation layer 6, replacement opening 61 and insulation layer 7.

### Example 1

This example is basically as shown in FIG. 2: a heat patch capable of targeted drug delivery based on a Monroe effect includes a fixation layer 6, a heating layer 4 and a drug layer 3 that are arranged in sequence. As a reference, the fixation layer 6 in this example is made of non-woven material that is convenient in air permeability. The adhesive layer 2 is arranged on an edge of a side, facing the drug layer 3, of the fixation layer 6, and the adhesive layer 2 and the drug layer 3 are covered with release liners 1. The adhesive layer 2 is made of a pressure-sensitive adhesive, which can fix the heat patch on skin of an affected part of a patient, and further form a square pressure-sensitive adhesive-free space in the affected part of the patient for drug permeation and improving a therapeutic effect. The square pressure-sensitive adhesive-free space can also prevent the patient from being sensitive and itchy under influence of a temperature of the heating layer 4. In other examples, the fixation layer 6 may also be a combination of a bandage and buckles (that is, two ends of the bandage are provided with matching buckles), an elastic bandage or a hook and loop fastener, so as to fix the heating layer 4 and the drug layer 3 on the affected part of the patient.

The heating layer 4 includes a heating sleeve 41 located peripherally and a heating assembly 42 located inside. The heating assembly 42 is any of an electric heating sheet, a magnetic heating sheet or an iron powder vermiculite heating bag. As a reference, the heating assembly 42 in this example is the iron powder vermiculite heating bag. The iron powder vermiculite heating bag may be formed by filling a cloth bag with powder such as iron powder vermiculite. The iron powder vermiculite heating bag can react with oxygen in air to release heat.

One or more heat collection cavities 43 that are provided with openings facing the drug layer are arranged in a side, close to the drug layer 3, in the heating layer 4. The heat collection cavity 43 has a shape of any or a combination of a semicircle, a cone and a semi-elliptic shape. The ratio between a height h and a diameter d of the heat collection cavity 43 is 0.3-1, and the diameter d≤10 cm. The heat collection cavity is made of any of stainless steel, high-carbon steel, engineering plastics, memory alloys and non-woven fiber elastic cotton. As a reference, in this example, a number of the heat collection cavity 43 is one, and the heat collection cavity is made of the memory alloy. The heat collection cavity 43 is located in a middle of the heating layer 4. The heat collection cavity 43 has a shape of the cone, with a height h of 6.93 cm and a diameter d of 8 cm, and the ratio of the height h to the diameter d is 0.87. The heat collection cavity 43 collects heat in the heating layer 4 into a bunchy shape and then transfers same to the drug layer 3 in a direction of an opening, so as to implement targeted treatment with a drug. A reference is made to a “Monroe effect” for a heat collection process of the conical heat collection cavity 43, which will not be repeated herein. The drug layer 3 is any or a combination of drug powder, a pill, ointment and a drug liquid. In this example, the drug layer 3 is made of multi-layer non-woven fabrics, and ointment is contained at a position, corresponding to the heat accumulation cavity 43, in a center of the medicinal layer 3. As a reference, in this example, the ointment is Cheezheng Sprain Relieving Plaster, and has a circle shape matching a shape of the opening of the heat collection cavity 43.

This solution further provides a preparation method of the heat patch capable of targeted drug delivery based on a Monroe effect. The preparation method includes: an iron powder vermiculite heating bag (heating assembly 42) is put into a heating sleeve 41 to form a heating layer 4. A fixation layer 6, the heating sleeve 41 and a drug layer 3 are bound into a whole, then a side, facing the drug layer 3, of the fixation layer 6 is coated with a pressure-sensitive adhesive, and then a release liner 1 is stuck on the pressure-sensitive adhesive to seal the drug layer 3 and the fixation layer 6 to form a heat patch. A packaging bag packs the heat patch, and the heat patch product is formed after vacuum packaging.

This solution further provides a use method for a heat patch capable of targeted drug delivery based on a Monroe effect. The use method includes:
(1) At a preparation stage: a heat patch is taken out from a packaging bag and a release liner 1 is torn off from a surface of the heat patch.
(2) At a fixation stage: a drug layer 3 is fixed to an affected part of a patient for treating the affected part of the patient. In this process, air enters a heating layer 4 from gaps in non-woven fabrics of a fixation layer 6, and an iron powder vermiculite heating bag comes into contact with and reacts chemically with oxygen in the air to release heat. The heat is collected into a bunchy shape by a heat collection cavity 43 and then transferred to ointment in a direction of an opening. The ointment is in a solidified state at a room temperature. The heat is collected at the ointment through the heat collection cavity, and the ointment is heated to a partially melted or semi-melted state, can conveniently come into contact well with skin and permeate into an affected part, and directly reach a focus. In this way, targeted drug delivery of the ointment is implemented, a therapeutic effect is improved, the heat can be effectively collected, a heat loss is avoided and energy utilization efficiency is improved. In addition, the heated plaster also helps to open pores, and effectively promotes drug absorption accordingly.

Examples 2-8 and Comparative Examples 1-8 are basically the same as Example 1, and differences mainly lie in a shape and a size of the heat collection cavity. The differences in the shape and size of the heat collection cavity in Examples 1-8 and Comparative Examples 1-8 are shown in Table 1 and FIG. 3. FIG. 3A shows a conical heat collection cavity, FIG. 3B shows a hemispherical heat collection cavity, FIG. 3C shows a semi-elliptic heat collection cavity, FIG. 3D shows a combined conical heat collection cavity, FIG. 3E shows a cylindrical heat collection cavity, and arrows indicate a direction of an opening of the heat collection cavity.

Experimental example 1 Heat collection effects of heat collection cavities that have different shapes and sizes

The applicant tests the heating effects of the heat patches of Examples 1-8 and Comparative Examples 1-8. By testing time (that is, heating time) required for plaster heating from a normal temperature to 40° C., a stable temperature (that is, a temperature of the heating layer) on a surface of the heating layer, a highest temperature on a surface of the plaster, and time (temperature keeping time) for keeping a high temperature after the plaster reaches the highest temperature, the heat collection effects of central heat collection cavities that have different shapes and sizes on the heat of the heating layer are comprehensively evaluated. The test results are shown in Table 1.

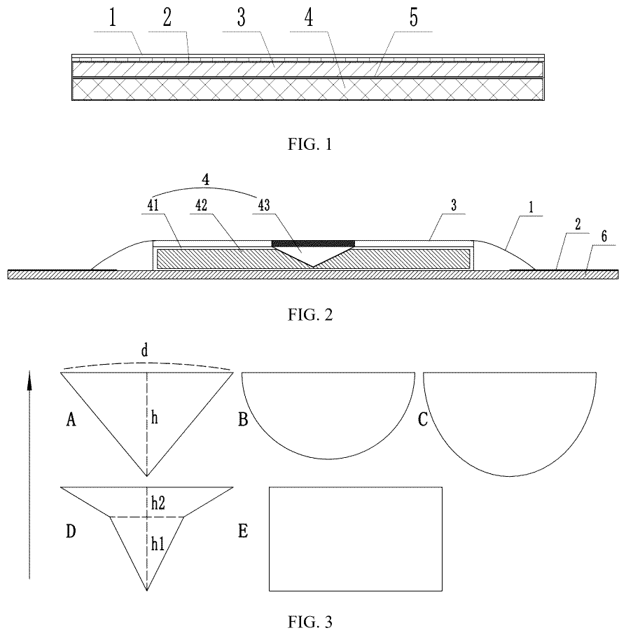

TABLE 1
Differences in shapes and sizes of heat collection cavities
in heat patches of Examples 1-8 and Comparative Examples 1-8

| | | | Diameter | | | | | |
|---|---|---|---|---|---|---|---|---|
| | | Height | of a | | | Temperature | Highest | |
| | Shape of | of a heat | heat | Ratio of | | of | temperature | |
| | a heat | collection | collection | the height | | a | on a | Temperature |
| | collection | cavity | cavity | h to the | Heating | heating | surface of a | keeping |
| Example | cavity | h/cm | d/cm | diameter d | time/min | layer/° C. | plaster/° C. | time/° C. |
| Example 1 | Cone | 6.93 | 8 | 0.87 | 10.1 | 46.1 | 57.9 | 10.2 |
| Example 2 | | 8.66 | 10 | 0.87 | 10.5 | 46.4 | 57.7 | 10.7 |
| Example 3 | | 2.5 | 5 | 0.50 | 10.6 | 46.9 | 56.6 | 10.5 |
| Example 4 | | 5 | 10 | 0.50 | 9.9 | 46.5 | 56.5 | 9.8 |
| Example 5 | | 3 | 10 | 0.30 | 12.9 | 46.1 | 55.3 | 8.9 |
| Example 5 | Hemisphere | 5 | 10 | 0.50 | 11.7 | 46.4 | 55.9 | 9.4 |
| Example 6 | Semi- | 3 | 10 | 0.30 | 11.3 | 46.3 | 55.7 | 9.8 |
| | elliptic | | | | | | | |
| | shape | | | | | | | |
| Example 7 | Combined | h1 = 3, | 10 | 0.45 | 10.5 | 46.7 | 58.9 | 9.7 |
| | cone | h2 = 1.5 | | | | | | |
| Example 8 | | h1 = 3, | 10 | 0.45 | 10.2 | 46.3 | 58.1 | 9.3 |
| | | h2 = 1.5 | | | | | | |
| Comparative | — | Heat patch as shown in FIG. 1 | | | 11.1 | 46.3 | 48.9 | 8.0 |
| example 1 | | | | | | | | |
| Comparative | Cone | 6 | 12 | 0.5 | 9.9 | 45.7 | 52.4 | 8.2 |
| example 2 | | | | | | | | |
| Comparative | | 1.3 | 10 | 0.13 | 12.7 | 45.2 | 52.8 | 8.2 |
| example 3 | | | | | | | | |
| Comparative | | 6 | 5 | 1.2 | 11.3 | 46.1 | 53.4 | 8.9 |
| example 4 | | | | | | | | |
| Comparative | Hemisphere | 6 | 12 | 0.50 | 11.5 | 46.5 | 52.7 | 8.5 |
| example 5 | | | | | | | | |
| Comparative | Semi- | 3.6 | 12 | 0.30 | 11.5 | 45.9 | 52.3 | 8.3 |
| example 6 | elliptic | | | | | | | |
| Comparative | shape | 2.1 | 10 | 0.21 | 12.2 | 46.6 | 51.4 | 8.3 |
| example 7 | | | | | | | | |
| Comparative | Cylinder | 5 | 10 | 0.50 | 14.2 | 46.5 | 51.1 | 8.2 |
| example 8 | | | | | | | | |

The experimental data show that the heating time, the temperature and the keeping time of ointment in the heat patches that have different shapes and sizes are tested, and it is found that the heat collection cavity that has a shape of any or a combination of the cone, the hemisphere and the semi-elliptic shape can effectively collect heat, the surface temperature of the plaster can be effectively improved and a therapeutic effect can be improved. Specifically, compared with a heat patch that is not provided with a heat collection cavity in Comparative Example 1, and has low heat utilization efficiency due to the highest temperature of ointment of 48.9° C., in Examples 1-8 of this solution, the heat collection cavities that have the different shapes and sizes can effectively collect heat, and when the highest surface temperature of ointment is raised to 55.3° C.-58.9° C., the heat collection cavity obtained by combining two-layer cones (such as Examples 7 and 8) has an optimal heat collection effect, and the highest surface temperature of the ointment reaches as high as 58.9° C. In this way, targeted drug delivery of the ointment is effectively implemented, and the therapeutic effect is improved. The temperature satisfies the requirement that “the highest temperature of a patch that directly comes into contact with human skin should not be higher than 60° C.” defined in 5.8.1 of the industry standard YY 0060-2018 Hot Compress Sticker (Bag).

By comparison, it is found that when the diameter of the heat collection cavity is less than 10 cm, the heat collection effects of the heat collection cavities has little differences when the heat collection cavities are scaled down, as shown in Examples 1 and 2 and Examples 3 and 4. However, if the ratio of the height h to the diameter d of the heat collection cavity is less than 0.3 (as shown in Comparative examples 3 and 7) or greater than 1 (as shown in Comparative example 4), the heat collection effect of the heat collection cavity on the heat of the heating layer is relatively reduced, which is manifested in a low highest surface temperature of the ointment. However, if the diameter of the heat collection cavity is too large (for example, the heat collection cavities have a diameter of 12 cm in Comparative Examples 2, 5 and 6), the highest surface temperature of the plaster will be reduced because of the excessively large heat collection cavity. However, if the heat collection cavity is designed as a cylinder (as shown in Comparative Example 8), even if the ration of the height to the diameter is 0.3-1 and the diameter d≤10 cm, a heat transfer speed can be reduced because a heat transfer surface is perpendicular to a surface of the ointment. The heat collection effect can be increased, it will take a long time for heating the ointment to 40° C. (the heating time is 14.2 min), the highest surface temperature of the ointment will be relatively low (specifically 51.1° C.), and the heat loss is caused accordingly.

Experimental example 2 Therapeutic Effects of heat patches that have different heat collection cavities

With Cheezheng Sprain Relieving Plaster as an example, the applicant evaluates the therapeutic of the heat patches in Example 1 and Comparative Example 1.
1. Conditions of Patients

The patients suffer a traumatic fracture, sprain and contusion, muscle swelling and pain, and have local clinical manifestations such as redness, swelling, heat and pain.

There were 122 patients. There were 67 males and 55 females. There were 34 male patients and 27 female patients in an experimental group and 33 male patients and 28 female patients in a control group.
2. Acceptance Criteria

There were 122 patients who volunteered to participate in the tests. The patients were intact and undamaged in local skin, had no abnormality in blood routine examination, liver functions, renal functions and coagulation mechanisms, and had no history of drug allergy.
3. Exclusion Criteria

A patient who fails to satisfy the acceptance criteria, has poor communication capacity, or rejects treatment, and pregnant women.
4. Treatment Methods

Experimental group: the heat patch of Example 1 of this solution was applied to an affected part of the patient for 6 hours a day and 7 consecutive days.

Control group: a heat patch was formed in Comparative example 1 by sticking an existing heat patch to a back of Cheezheng Sprain Relieving Plaster, and the heat patch was stuck to the affected part for 6 hours a day and for 7 consecutive days.
5. Therapeutic Effect Evaluation Indexes

The therapeutic effect evaluation is shown in Table 2, and the therapeutic effects of the experimental group and the control group are shown in Table 3.

TABLE 2
Therapeutic effect evaluation

| | Completely | Partially | Slightly | Completely |
|---|---|---|---|---|
| Type | relieved | relieved | relieved | ineffective |
| Effect | The | The | The redness | No change |
| | congestion, | congestion, | and swelling | at all |
| | redness and | redness and | are obviously | |
| | swelling | swelling are | reduced, | |
| | disappear | obviously | and the scope | |
| | completely | reduced | of congestion is | |
| | | | smaller than | |
| | | | before | |

TABLE 3
Therapeutic effects of the experimental group and the control group

| | Number | | | | | |
|---|---|---|---|---|---|---|
| | of | Completely | Partially | Slightly | Completely | |
| Group | patient | relieved | relieved | relieved | ineffective | Efficient |
| Experimental group | 61 | 37 | 12 | 12 | 0 | 100.00% |
| Control group | 61 | 22 | 11 | 17 | 11 | 81.97% |

The experimental data show that according to the heat patch in this solution, the heat in the heating layer can be effectively collected by arranging the heat collection cavity, the highest temperature on the surface of the ointment is raised, and the ointment can fit skin of the affected part of the patient to a greater extent in a semi-melted state. In this way, the skin of the affected part of the patient can be warmed and pores can be opened for the convenience of permeation of to the ointment. In addition, the continuous high temperature of the ointment can also speed up the permeation speed of ointment, and the therapeutic effect of the ointment can be improved accordingly. Specifically, compared with adjuvant treatment (the effective rate is only 81.97%) using a heat patch that is not provided with a heat collection cavity in Comparative Document 1, the heat patch in Example 1 of this solution can effectively improve the therapeutic effect for patients suffering the traumatic fracture, sprain and contusion, muscle swelling and pain, and the effective rate is as high as 100%.

### Example 9

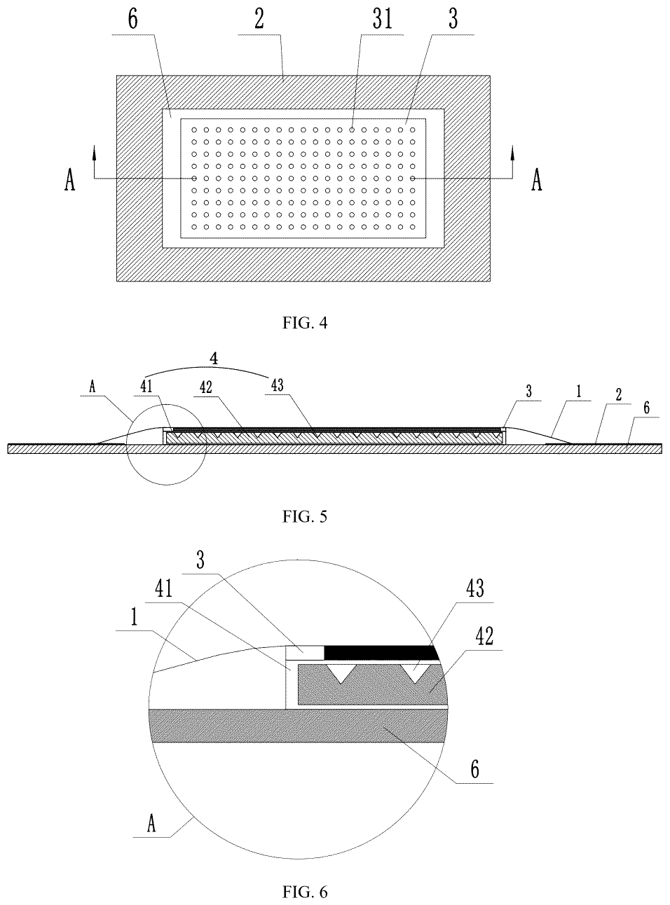

In order to further improve sticking convenience of a heat patch, a difference between this solution and Example 1 lies in that, as shown in FIGS. 4-6, a number of heat collection cavities 43 is several, a shape the heat collection cavities is conical, and several heat collection cavities 43 are distributed in an array. In this example, each heat collection cavity 43 arranged can collect heat in the heating layer 4 into a bunchy shape and then transfer same to a drug in the drug layer 3 in a direction of an opening. In this way, the drug can permeate into the affected part of the patient in a spotted way and reach the focus directly, and targeted drug delivery can be implemented. The heat patch of this example is particularly suitable for adjuvant treatment of an affected part with dispersive acupuncture points.

### Example 10

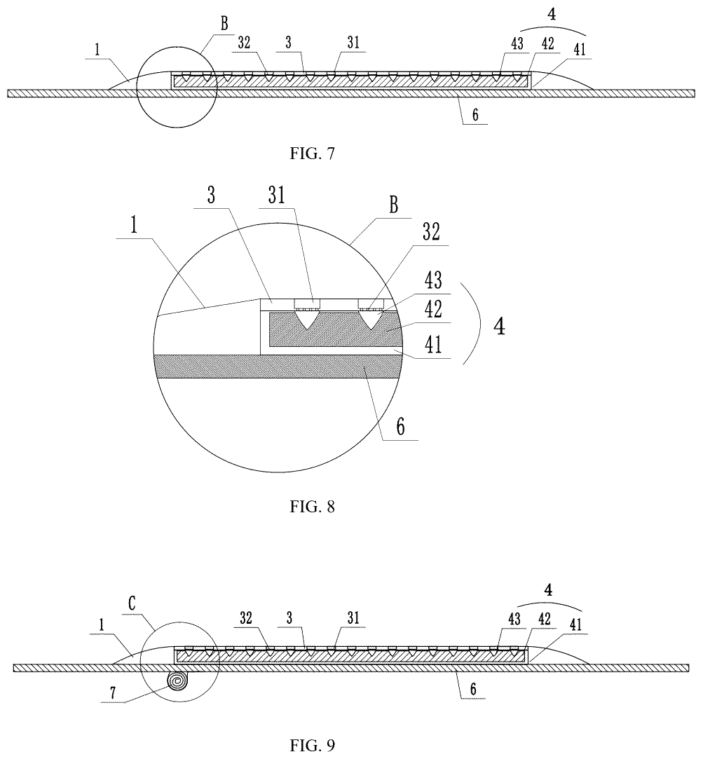

In order to satisfy a therapeutic effect of a patient and further reduce a use amount of a drug, as shown in FIGS. 7-8 of this example, a drug layer 3 is provided with a drug cavity 31 that is configured to contain the drug corresponding to a heat collection cavity 43, and the drug cavity 31 is a blind hole with an opening far away from the heating layer 4. A heat dissipation hole 32 is provided between the drug cavity 31 and the heat collection cavity 43. In this solution, the drug that may be contained in the drug cavity 31 is any or a combination of powder, a pill, ointment and a drug liquid. As a reference, the drug layer 3 in this solution is made of a multi-layer non-woven material, and the drug cavity 31 is filled with a drug in advance, or after heat patches are produced with empty cavities and sold, the cavities are filled with appropriate drugs by patients according to illness. Specifically, in this example, the drug cavity 31 is an empty cavity, and the patient will fill the empty cavity with appropriate drugs according to illness after purchasing the heat patch products before use.

This solution further provides a use method for a heat patch capable of targeted drug delivery based on a Monroe effect. The use method includes:
(1) At a preparation stage: a heat patch is taken out from a packaging bag and a release liner 1 is torn off from a surface of the heat patch.
(2) At a drug filling stage: the patient selects the appropriate drug according to the illness, and fills drug cavities 31 with drugs, such that all the drug cavity 31 are filled with drugs.
(3) At a sticking stage: a drug layer 3 is fixed to the affected part of the patient for treating the affected part of the patient. In this example, the drug is filled in a spotted manner, such that the use amount of drug is effectively reduced and a cost is reduced. In addition, the drug layer 3 made of multi-layer non-woven fabrics can effectively isolate the heating layer 4 from direct contact with the skin of the patient, such that cryogenic burn risk is effectively reduced.

### Example 11

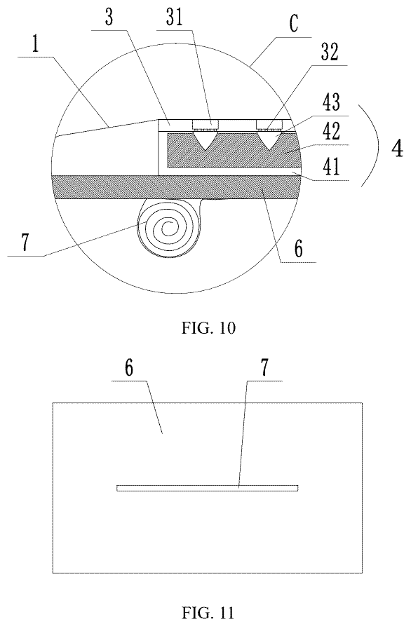

In order to further reduce the cryogenic burn risk of the heat patch, a difference between this solution and Example 2 lies in that, as shown in FIGS. 9-10, an insulation layer 7 is arranged on a side, far away from a drug layer 3, of a fixation layer 6, the insulation layer 7 includes a fixed end connected to the fixation layer 6 and a windable free end, and the fixation layer 6 is provided with a fixation member that detachably fixes the free end of the insulation layer 7 near the fixed end. As a reference, the insulation layer 7 in this example is made of a plastic film, and the fixation member is a hook and loop fastener.

In this example, in an early use stage of the heat patch, the insulation layer 7 is wound to facilitate air in contact an iron powder vermiculite heating bag through the fixation layer 6 and a heating sleeve 41, such that contents of the iron powder vermiculite heating bag react with oxygen in the air to release heat, and the heat is transferred to the drug in a bunchy shape for targeted treatment. However, when a heating assembly 42 is too hot and makes the patient uncomfortable (for example, some temperature-sensitive patients have poor temperature tolerance), winding fixation of the insulation layer 7 by the fixation member is released, the insulation layer 7 is unwound and closely attached to an outer side surface of the heat patch, and in this case, the insulation layer 7 is in an unwound isolation state. In this way, the oxygen in the air can be isolated effectively from entering the heating layer 4, the chemical exothermic reaction of the iron powder vermiculite heating bag can be blocked, and heat release is reduced and physical perception of the patient is relieved. According to this solution, the application scope of the heat patch is effectively expanded by arranging the insulation layer 7.

### Example 12

In order to further improve stability of heat release of the heat patch, a difference between this solution and Example 1 lies in that a heating assembly 42 is an electric heating sheet. The electric heating sheet is connected to a power connection line, and the other end of the power connection line is a plug or USB interface. In this example, a number of heat collection cavities is one, and the heat collection cavity is located in a middle of a heating layer. In this solution, the plug or the USB interface is connected to a socket or a personal computer (PC) end of a computer, such that stable energization and heating can be implemented, and the heat release stability of the heat patch can be effectively improved. In addition, even if a heat patch with lower power is used, a heat collection cavity located in a middle of a heating layer (also located in a middle of the heat patch) can effectively collect surrounding heat, targeted drug delivery is implemented and a therapeutic effect is improved.

### Example 13

In order to further prolong a service life of the heat patch and reduce a treatment cost, a difference between this solution and Example 1 lies in that as shown in FIG. 11, a heating sleeve 41 and a fixation layer 6 are provided with replacement openings 61, the replacement opening 61 is located in a middle of a side, far away from a drug layer 3, of the fixation layer 6, and an adhesive layer 2 is a traceless adhesive tape. In this example, if an iron powder vermiculite heating bag no longer releases heat, the iron powder vermiculite heating bag can be taken out from the replacement opening 61. In addition, the adhesive layer 2 is arranged as the traceless adhesive tape, such that a patient can conveniently tear off the heat patch and stick same again after changing the drug. In the two ways, the heat patch can be reused and the service life of the heat patch can be effectively prolonged.

The above examples are merely the examples of the present disclosure, and common general knowledge including specific technical solution and/or features in the solution is not described herein. It should be noted that for those skilled in the art, several modifications and improvements can be made without departing from the technical solution of the present disclosure, and such modifications and improvements should also be considered to fall within the protection scope of the present disclosure without influence on implementation effects and patent of the present disclosure and practicability of the patent. The protection scope required by the present disclosure shall be subject to the contents of the claims, and explanation of the specific embodiments, etc. in the description can be used to explain the contents of the claims.

What is claimed is:

1. A heat patch capable of targeted drug delivery based on a Monroe effect, comprising: a fixation layer, a heating layer and a drug layer that are arranged in sequence, wherein a heating assembly is arranged in the heating layer, a heat collection cavity that is provided with an opening facing the drug layer is provided in a side, close to the drug layer, in the heating layer, and a drug is contained at a portion, corresponding to the opening of the heat collection cavity, in the drug layer.

2. The heat patch capable of targeted drug delivery based on a Monroe effect according to claim 1, wherein the heat collection cavity has a shape of any or a combination of a semicircle, a cone and a semi-elliptic shape.

3. The heat patch capable of targeted drug delivery based on a Monroe effect according to claim 2, wherein the heat collection cavity is made of any of stainless steel, high-carbon steel, engineering plastics, memory alloys and non-woven fiber elastic cotton.

4. The heat patch capable of targeted drug delivery based on a Monroe effect according to claim 3, wherein the ratio between a height h and a diameter d of the heat collection cavity is 0.3-1, and the diameter d≤10 cm.

5. The heat patch capable of targeted drug delivery based on a Monroe effect according to claim 4, wherein the drug layer is provided with a drug cavity that is configured to contain the drug corresponding to the heat collection cavity, the drug cavity is a blind hole, the drug cavity is located in a side, far away from the heating layer, of the drug layer, and the drug contained in the drug cavity is any or a combination of drug powder, a pill, ointment and a drug liquid.

6. The heat patch capable of targeted drug delivery based on a Monroe effect according to claim 5, wherein a heat dissipation hole is provided between the drug cavity and the heat collection cavity.

7. The heat patch capable of targeted drug delivery based on a Monroe effect according to claim 6, wherein the heating layer comprises a heating sleeve located peripherally and the heating assembly located inside, the heating assembly is any of an electric heating sheet, a magnetic heating sheet or an iron powder vermiculite heating bag, and the heating sleeve and the fixation layer are provided with replacement openings.

8. The heat patch capable of targeted drug delivery based on a Monroe effect according to claim 7, wherein the heating assembly is the iron powder vermiculite heating bag, an insulation layer is arranged on a side, far away from the drug layer, of the fixation layer, the insulation layer comprises a fixed end connected to the fixation layer and a windable free end, and the fixation layer is provided with a fixation member that detachably fixes the free end of the insulation layer near the fixed end.

9. The heat patch capable of targeted drug delivery based on a Monroe effect according to claim 8, wherein an adhesive coating and a release liner are sequentially arranged on a side, facing the drug layer, of the fixation layer.

10. The heat patch capable of targeted drug delivery based on a Monroe effect according to claim 9, wherein one or several heat collection cavities are provided, one heat collection cavity is located in a middle of the heating layer of the heat patch, and the several heat collection cavities are distributed in a matrix.
